(19) United States
(12) Patent Application Publication
(10) Pub. No.: US 2026/0262930 A1
(43) Pub. Date: Sep. 10, 2026

(54) AIRWAY EXCHANGE BRONCHOSCOPE CONSTRUCTION

(71) Applicant: University of Southern California, Los Angeles, CA (US)

(72) Inventors: Roger Boles, Los Angeles, CA (US); Wihan Kim, Los Angeles, CA (US)

(21) Appl. No.: 19/164,913

(22) PCT Filed: Mar. 13, 2024
(86) PCT No.: PCT/US2024/019753

§ 371 Date: Sep. 12, 2025

Related U.S. Application Data
(60) Provisional application No. 63/451,903, filed on Mar. 13, 2023.

(51) Int. Cl. A61B 1/267; A61B 1/00; A61B 1/05; A61M 16/04

(52) U.S. Cl. CPC A61B 1/2676; A61B 1/00043; A61B 1/00128; A61B 1/00135; A61B 1/00147; A61B 1/05; A61M 16/0488; A61M 2205/502; A61M 2205/583; A61M 2210/1025

(57) ABSTRACT

Airway exchange bronchoscope constructions comprise a bronchoscope having a body and a tube extending outwardly therefrom, and comprise an airway exchange catheter including a tube that is configured to extend concentrically over the bronchoscope tube. An attachment mechanism is configured to releasably connect the bronchoscope with the airway exchange catheter when the bronchoscope tube is disposed within the airway exchange catheter tube. The bronchoscope includes a camera at an end having a field of view out of an adjacent end of the airway exchange catheter. Configured in this manner, the airway exchange bronchoscope construction enables one through use of a visual display to visually guide placement and positioning of the airway exchange catheter in an airway of a human patient.

### CROSS-REFERENCE TO RELATED APPLICATIONS

This application claims the benefit and priority to U.S. Provisional Patent Application No. 63/451,903 filed Mar. 13, 2023, entitled “AIRWAY EXCHANGE BRONCHOLARYNGOSCOPE CONSTRUCTION,” the entire content of which is hereby incorporated by reference in its entirety.

Airway exchange constructions, assemblies, devices, systems, and methods for using the same as disclosed herein are configured for use within an airway of a human body and, more particularly, are specially constructed and/or configured to enable and ensure correct insertion and use of an airway exchange catheter in an airway of a human body.

### BACKGROUND

Bronchoscopy is a procedure that allows physicians to examine the lungs and air passages. During bronchoscopy, a thin tube (bronchoscope) is passed through the nose or mouth, down the throat and into the lungs, and includes a camera at a leading end that provides an image for viewing. Bronchoscopes can also be inserted into airway tubes, such as endotracheal tubes or tracheostomy tubes, for evaluation of the tubes themselves as well as the airway. Common reasons for needing bronchoscopy are a persistent cough or infection. Bronchoscopy can also be used to obtain samples of mucus or tissue, to remove foreign bodies or other blockages from the airways or lungs, or to provide treatment for respiratory issues.

Conventional airway exchange catheters are devices used to provide a reliable passage into a patient's airway and lungs while exchanging one airway tube (e.g. endotracheal tube, tracheostomy tube) for another. During insertion of such airway exchange catheter, it is important that the airway exchange catheter be properly inserted into the patient's airway. There may be certain instances, e.g., where the patient has a fistula or hole between the patient's airway and adjacent esophagus, that may make proper insertion of the airway exchange catheter into the patient's airway challenging. In such an event, it is important that during insertion of such conventional airway exchange catheter that the airway exchange catheter be properly inserted into the patent's airway and not into the patient's esophagus. Moreover, blind insertion of the airway exchange catheter can cause traumatic damage to anatomic structures in the airway.

Current standard practice for procedures in which one airway tube is exchanged for another also requires a surgeon or physician to switch between using a bronchoscope and an airway exchange catheter without the ability to use both simultaneously. The need to transition between tools in this way increases procedural steps and time creating an increased risk to patient safety during the procedure.

It is, therefore, desired that a device be constructed in a manner that provides an improved degree of placement of an air exchange catheter into a patient's airway. It is also desired that such device be relatively easy to use and provide a high degree of certainty regarding proper insertion into a patient's airway during use. Finally, it is desired that a device be specifically configured to minimize steps and time associated with airway exchanges procedures.

### SUMMARY

Airway exchange bronchoscope constructions as disclosed herein generally comprise a bronchoscope comprising a body and a tube extending outwardly therefrom, and an airway exchange catheter comprising a tube that is configured to extend concentrically over the bronchoscope tube. In an example, the airway exchange catheter tube may be configured to extend along a substantial entirety of the bronchoscope tube when the airway exchange catheter is attached with the bronchoscope. In an example, airway exchange bronchoscope constructions as disclosed herein are may be part of a system comprising a video display connected thereto for purposes of enabling use of airway exchange bronchoscope constructions as disclosed herein to provide visual guidance and placement of the airway exchange catheter into an airway of a human patient.

An attachment mechanism is used to releasably connect or disconnect the airway exchange catheter to the bronchoscope. In an example, the attachment mechanism may be rotatable or comprise a rotatable element. In an example, the attachment mechanism may be configured to engage a feature of at least one of the bronchoscope and the airway exchange catheter, and/or the attachment mechanism may comprise a feature configured to engage at least one of the bronchoscope and the airway exchange catheter. In an example, the attachment mechanism may comprise comprises one or more magnetic elements configured to promote connection or attachment between the airway exchange catheter and the bronchoscope by magnetic force. In an example, the attachment mechanism may be configured to provide connection of the airway exchange catheter and the bronchoscope by ends of one or both of the airway exchange catheter and the bronchoscope being pushed into the attachment mechanism, and be disconnected by the ends of one or both of the airway exchange catheter and the bronchoscope being pulled from the attachment mechanism.

In an example, exchange bronchoscope constructions as disclose herein may be used for visually guiding placement of an airway inside of a human patient by inserting the airway exchange catheter into an airway of a patient. Wherein before or after the step of inserting, installing a tube of the bronchoscope inside the tube of the airway exchange catheter. Wherein the bronchoscope comprises a camera at an end that has a field of view or vision directed out of an end of the airway exchange catheter tube that is disposed in the airway. During the process or procedure, an image provided by the bronchoscope camera is viewed on a video display to visual guide and/or confirm a desired placement position of the airway exchange catheter in the airway of the patient. In an example, during such process or procedure, the tube of the bronchoscope is installed inside the tube of the airway exchange catheter before the airway exchange catheter is inserted into the airway of the patient. In an example, during such process or procedure, during the step of installing, the attachment mechanism is used to releasably connect the bronchoscope to the airway exchange catheter.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

These and other features and advantages of airway exchange bronchoscope constructions and methods for using the same as disclosed herein will be appreciated as the same becomes better understood by reference to the following detailed description when considered in connection with the accompanying drawings wherein:

FIGS. 1 and 2 are side perspective views of bronchoscopes that may be used with airway exchange bronchoscope constructions as disclosed herein;

FIG. 3 is a side perspective view of an airway exchange catheter that may be used with airway exchange bronchoscope constructions as disclosed herein;

FIGS. 4 and 5 are side perspective views of one embodiment of the airway exchange bronchoscope construction as disclosed herein;

FIGS. 6 and 7 are side perspective views of another embodiment airway exchange bronchoscope construction as disclosed herein;

FIG. 8 is a side exploded view of the embodiment of the airway exchange bronchoscope construction illustrated in FIGS. 6 and 7;

FIGS. 9 and 10 are distal end perspective views of an embodiment of an example airway exchange bronchoscope construction as disclosed herein;

FIG. 11 is a side perspective view of an airway exchange catheter as used with airway exchange bronchoscope constructions as disclosed herein;

FIGS. 12A to 12D are different views of an example attachment mechanism for airway exchange bronchoscope constructions as disclosed herein; and

FIGS. 13A to 13I are photographs illustrating example different steps for using an example airway exchange bronchoscope construction as disclosed herein.

### DETAILED DESCRIPTION

Airway exchange bronchoscope constructions as disclosed herein are specially configured to permit the combined use of a bronchoscope as connected with an airway exchange catheter to thereby enable precise insertion of the airway exchange catheter into a human patient's airway. A feature of such constructions as disclosed herein is the coupling together and/or attachment mechanism used to connect the airway exchange catheter with the bronchoscope that facilitates and permits ease of use, i.e., attachment and detachment of the airway exchange catheter therewith as better described below.

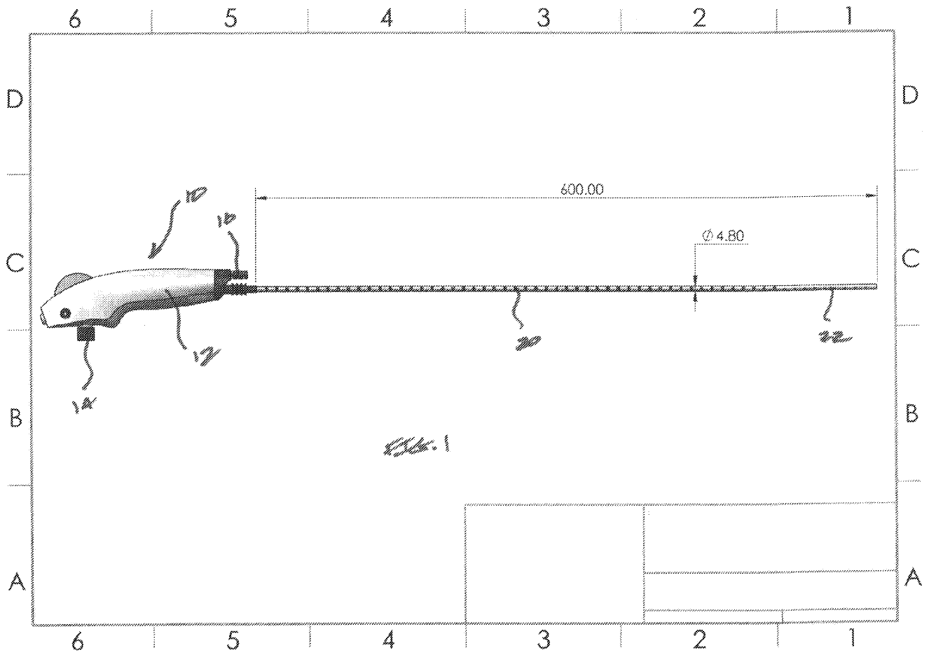

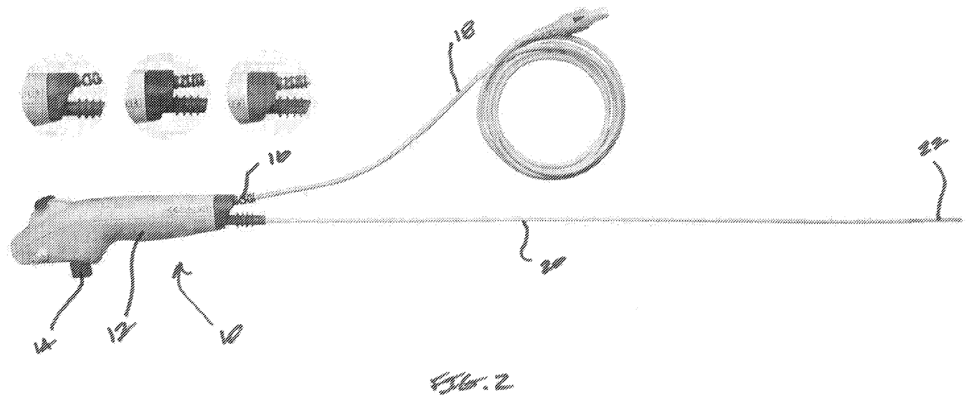

FIGS. 1 and 2 illustrate example bronchoscopes 10 that many be used with airway exchange bronchoscope constructions as disclosed herein. In an example, the bronchoscope 10 comprises a body 12 that may be configured for holding in a user's hand and that that includes one or more control features 14 that enable the user to operate one or more features of the bronchoscope. The bronchoscope 10 may include a connecter 16 that may be configured to connect with a cable 18 or the like for providing power to the bronchoscope 10 and/or for sending a video signal through the cable to a suitable video monitor (not shown) for viewing. The bronchoscope 10 comprises a long tube 20 extending from the body 12 that is configured having one or more features at an end section 22 of the tube 20 such as a camera, light, and the like known to be associated with bronchoscopes. In an example, the tube 20 may be flexible and/or the end section 22 of the tube may be flexible to permit a user to move the end radially as needed during use by operation of the control feature 14. While a certain example bronchoscope has been illustrated and described, it is to be understood that bronchoscopes other than that illustrated and described may be used to form airway exchange bronchoscope constructions as disclosed herein. Also, bronchoscopes as disclosed herein may also be referred to broncholaryngoscopes in this description. In an example, a bronchoscope such as one made by Ambu, e.g., a conventional distal-chip bronchoscope, may be suitable for use in forming airway exchange bronchoscope constructions as disclosed herein.

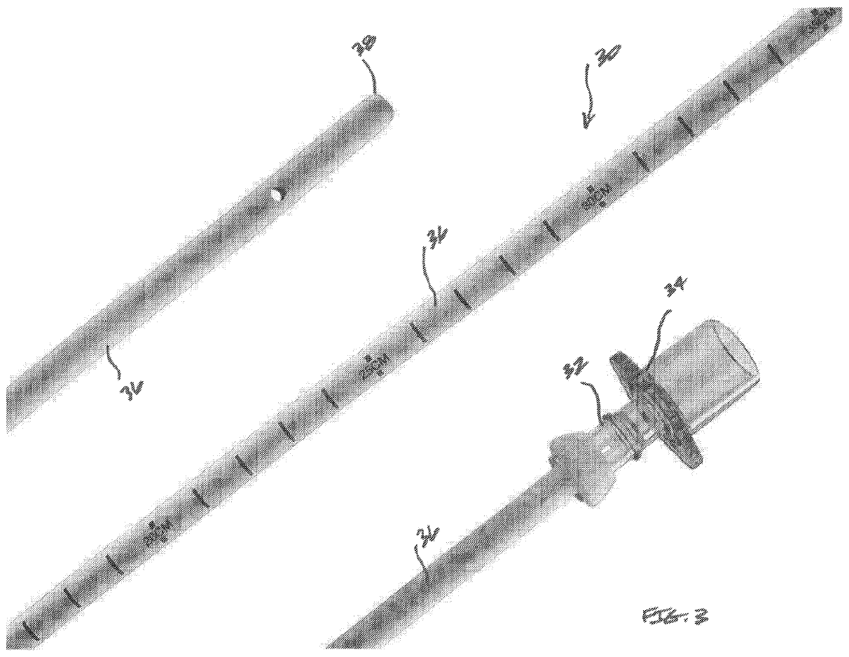

FIG. 3 illustrates an airway exchange catheter 30 that may be used with airway exchange bronchoscope constructions as disclosed herein. In an example, the airway exchange catheter comprises an attachment end 32 that is configured to accommodate attachment with an adaptor 34 configured for connection with a ventilator machine (not shown). The attachment end 32 is at one end of a hollow tube 36 that extends to an end 38 opposite the attachment end 32. Openings or holes 40 may be positioned radially adjacent the tube end 38 for purposes of providing and receiving air from the patient's lungs during use. While a certain example airway exchange catheter has been illustrated and described, it is to be understood that airway exchange catheters other than that illustrated and described may be used to form airway exchange bronchoscope constructions as disclosed herein. In an example, an airway exchange catheter such as one made by Cook Medical may be suitable for use in forming airway exchange bronchoscope constructions as disclosed herein. In a particular example, the airway exchange catheter may have an inner diameter of approximately 4.826 mm and be formed from PVC plastic chemical-resistant tube sleeving, and may have a length of approximately 57 cm.

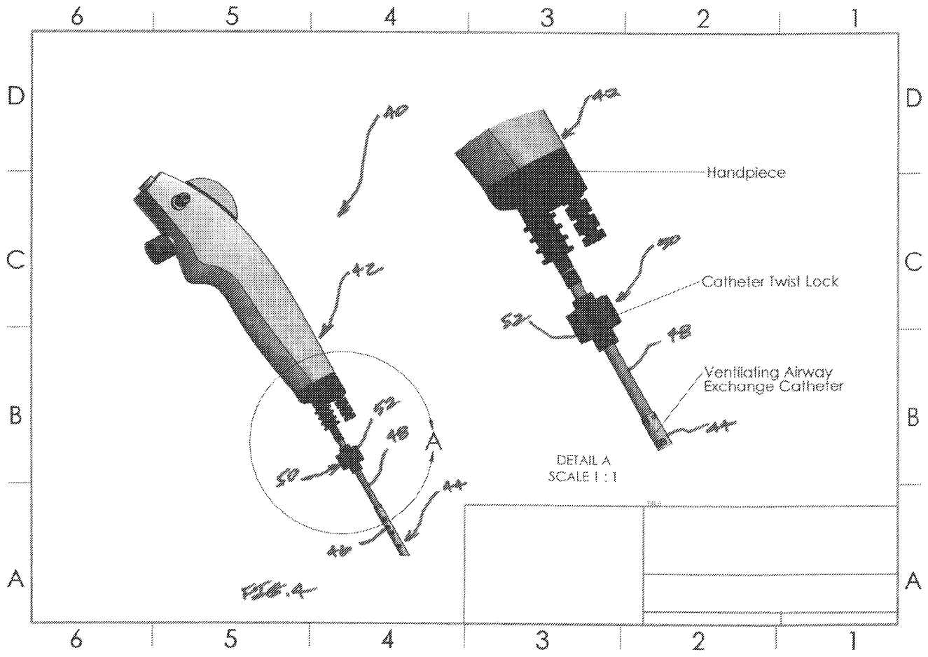

FIGS. 4 and 5 illustrate an example airway exchange bronchoscope construction 40 as disclosed herein comprising a bronchoscope 42 as combined with an airway exchange catheter 44, wherein the airway exchange catheter tube 46 is disposed concentrically over the bronchoscope tube 48. In this example embodiment, an attachment mechanism 50 is configured in the form of a twist lock element 52 that is attached to the bronchoscope adjacent an upper portion of the tube 48 and that is configured for rotational movement. With reference to FIG. 5, the twist lock element 52 is configured having an element, such as a projection or the like extending from within the twist lock element that is configured to register with and engage a surface feature of the airway exchange catheter tube 46 adjacent an end of the tube 46 adjacent the bronchoscope 42. FIG. 5 illustrates three different states of the attachment mechanism 50 (moving from left to right) from being in a locked state 54 with the attachment mechanism locking the airway exchange catheter with the bronchoscope (e.g., readied for insertion into a patient's airway), to being in an unlocked state 56 with the attachment mechanism being unlocked from the airway exchange catheter (e.g., in preparation for removal of the bronchoscope from the airway exchange catheter after insertion), to being in a detached state 58 with the attachment mechanism detached from the airway exchanged catheter (e.g., for removal of the bronchoscope from the airway exchange catheter to permit use of the airway exchange catheter). In this example, the attachment mechanism is configured to move from a locked to an unlocked state by rotation of the twist lock element. Alternatively, movement from a locked to an unlocked state may be provided by rotational movement of the airway exchange catheter relative to the attachment mechanism.

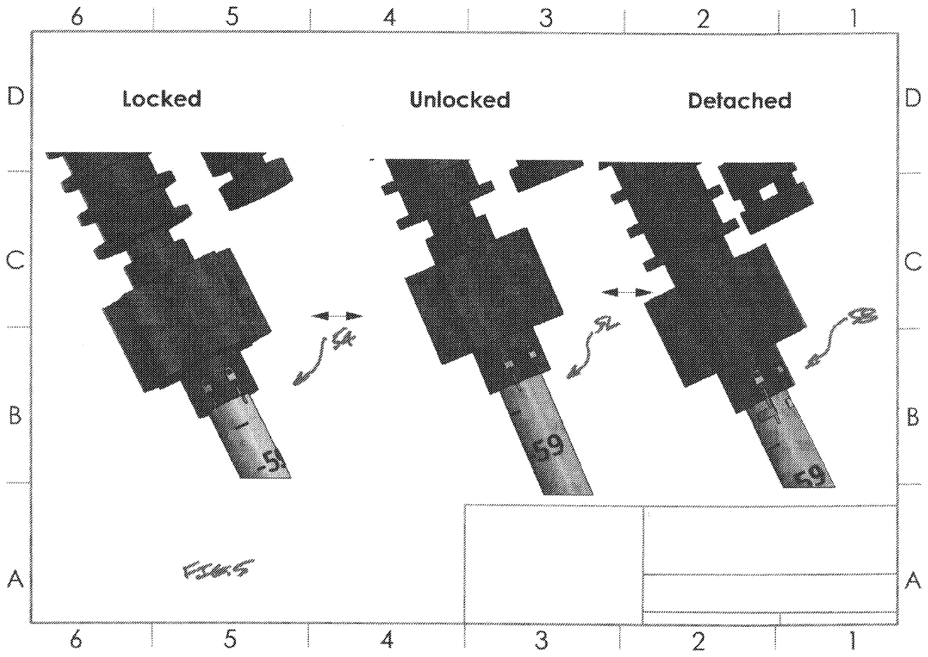

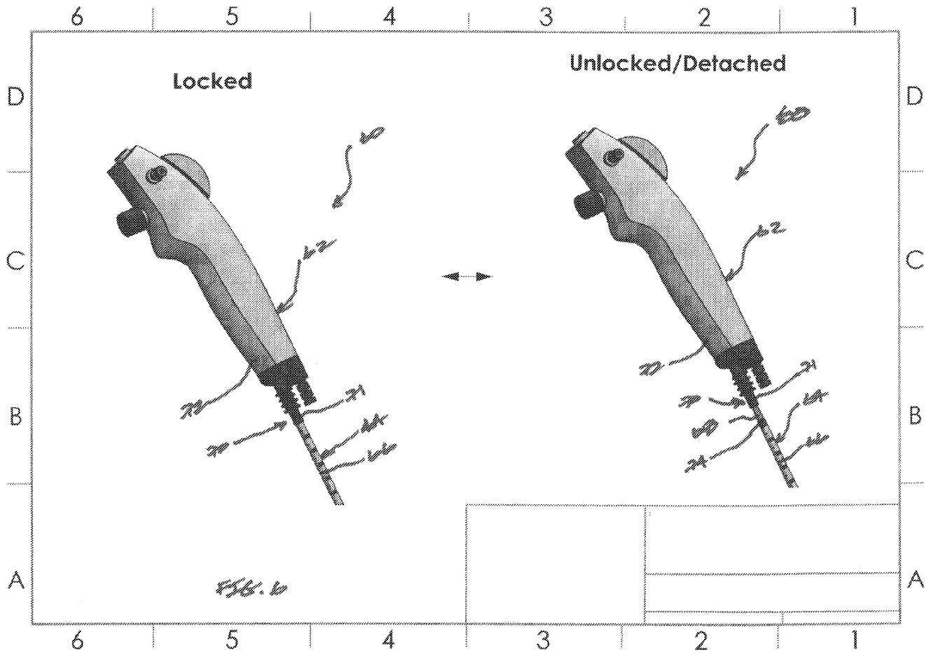

FIGS. 6 and 7 illustrate another example airway exchange bronchoscope construction 60 as disclosed herein comprising a bronchoscope 62 as combined with an airway exchange catheter 64, wherein the airway exchange catheter tube 66 is disposed concentrically over the bronchoscope tube 68. In this example embodiment, an attachment mechanism 70 is configured in the form a magnetic coupling, wherein one magnet 71 is attached to the bronchoscope adjacent the bronchoscope body 72 and another magnet 74 is attached adjacent an end of the airway exchange catheter tube 66 nearest the bronchoscope. Configured in this matter, the attachment mechanism 70 facilitates attachment and detachment between the bronchoscope and the airway exchange catheter by coupling and decoupling of the magnets 71 and 74 by axial movement of the airway exchange catheter respectively towards and away from the bronchoscope. In an example embodiment, the magnetic attachment mechanism is one that is configured to provide a desired locking attachment so that the airway exchange catheter does not become easily or inadvertently detached therefrom during insertion or use, and that also enables detachment by a user with the application of a desired force once the airway exchange catheter is inserted. FIG. 8 illustrates an exploded view of the magnets 71 and 74 of the magnetic attachment mechanism in an unattached or uncoupled state before respective attachment with the bronchoscope and the airway exchange catheter.

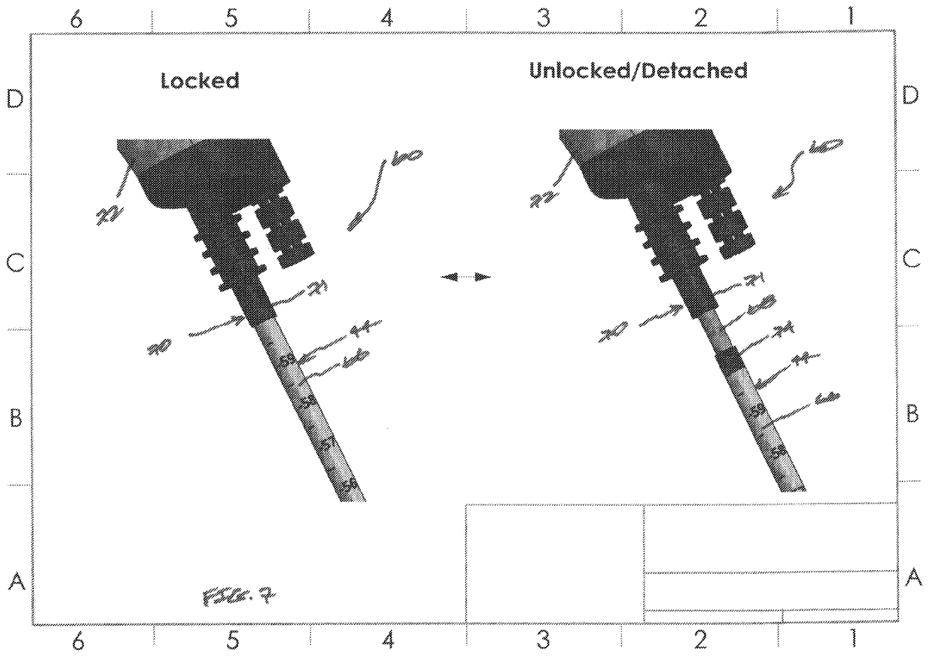

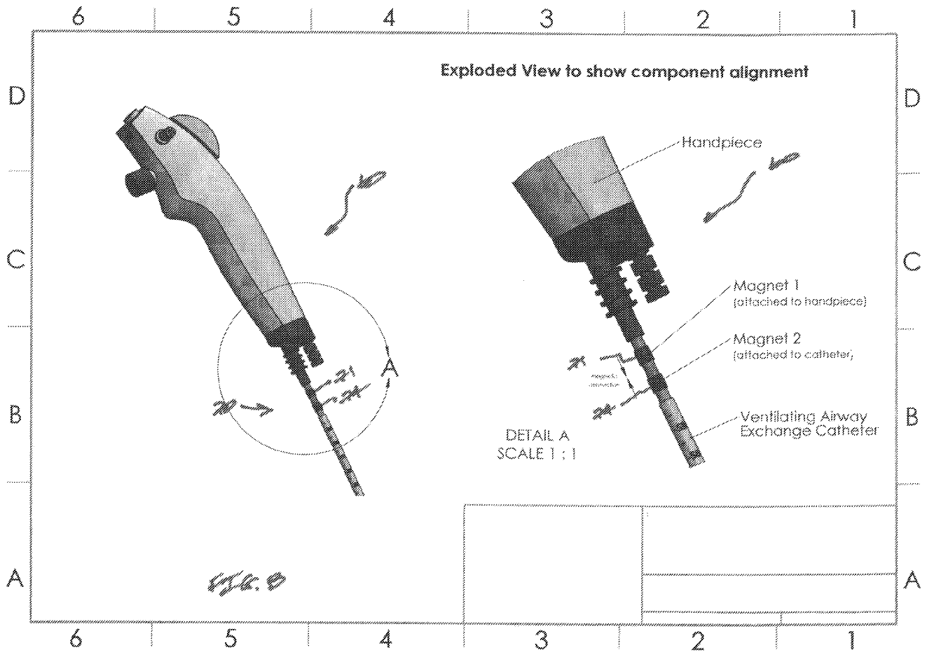

FIGS. 9 and 10 illustrate end views of an example airway exchange bronchoscope construction 80 as disclosed herein comprising an end 82 of the bronchoscope as projecting from an end 84 of the airway exchange catheter tube 86 surrounding the bronchoscope tube 88. In this example, the end of the bronchoscope 82 may include and not be limited to a camera 90, one or more angulation wires 92 to facilitate movement of the end of the bronchoscope, a suction element 94, and one or more lights 96. In an example, the end 82 of the bronchoscope may be surrounded by a more flexible section of the bronchoscope tube 88 to facilitate movement of the same. This is but one illustrated example of how the end of airway exchange bronchoscope constructions as disclosed herein may be configured and it is to be understood that airway exchange bronchoscope constructions as disclosed herein may be configured differently than as illustrated and that such other configurations are intended to be within the scope of airway exchange bronchoscope constructions as disclosed herein.

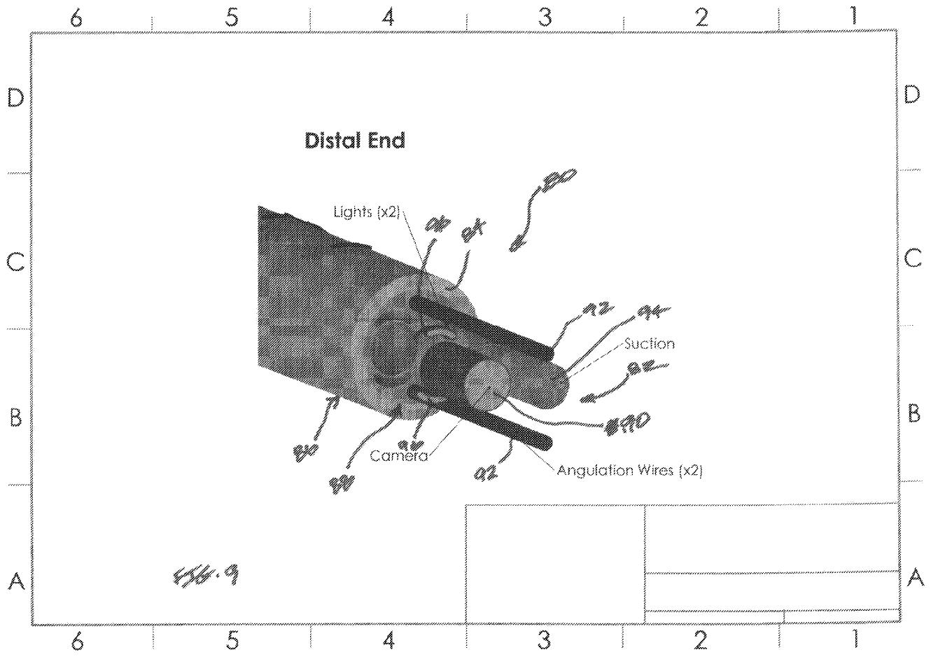

FIG. 11 illustrates an example airway exchange catheter 100 of the airway exchange bronchoscope construction as disclosed herein after the airway exchange bronchoscope construction has been inserted and the bronchoscope has been detached and removed therefrom. Moving from left to right, the airway exchange catheter 100 is shown in a detached state with an adapter 102 detached from an end 104 of the airway exchange catheter tube 106, and is shown an attached state with the adaptor 102 attached to the end of the airway exchange catheter tube 106. In an example, an attachment mechanism 106 is configured to facilitate attachment and detachment between the adaptor 102 and the end 104 of the airway exchange catheter. In an example, the attachment mechanism may be configured to utilize the same attachment feature of the airway exchange catheter used to facilitate attachment with the bronchoscope.

While certain types of mechanisms useful for attaching the airway exchange catheter with the bronchoscope have been described and illustrated for purposes of reference, it is to be understood that mechanisms other than those described and illustrated that are configured to perform the same or similar function, i.e., to facilitate attachment and detachment between the airway exchange catheter and bronchoscope are intended to be within the scope of airway exchange bronchoscope constructions as disclosed herein.

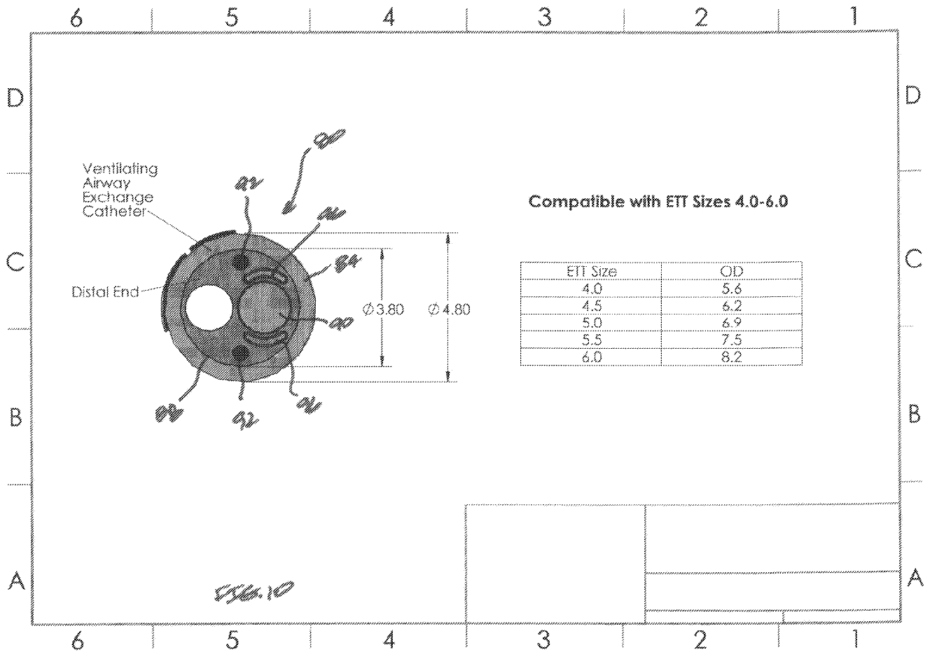

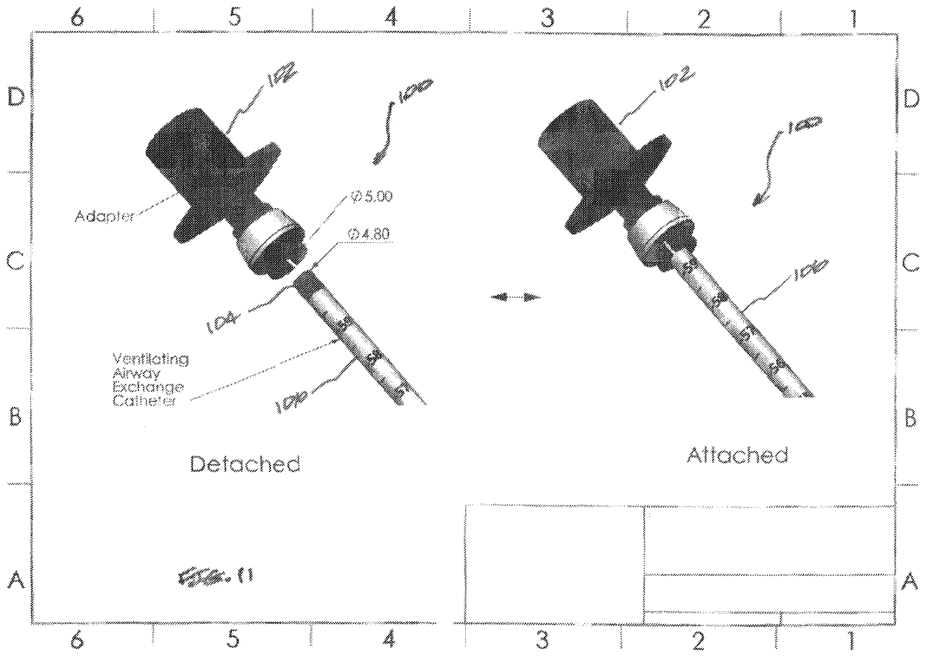

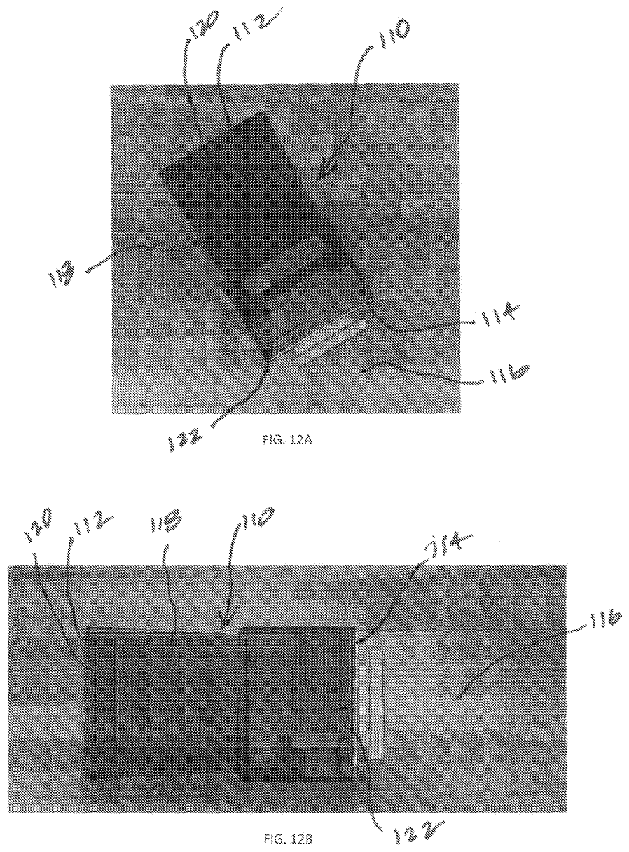

FIGS. 12A to 12D illustrate another example attachment mechanism 110 configured to facilitate attachment and detachment between the airway exchange catheter and bronchoscope. The example attachment mechanism 110 is configured to promote attachment and detachment at one end 112 with an end of a bronchoscope by respective push on and pull off action, and is configured to promote attachment and detachment at an opposite end 114 with an end of an airway exchange catheter 116 by respective push on and pull off action. In an example, the attachment mechanism 112 comprises a body or housing 118 formed from a structurally rigid material such as metal, plastic, or the like. Collars 120 and 122 are disposed within the body 118 at opposed ends 112 and 114 and are circular in shape and formed from an elastomeric material. In an example, the collars 120 and 122 are biased radially inwardly and are sized to both enable passage of respective ends of the bronchoscope and airway exchange catheter therein, e.g., by pushing action, and retain and provide a leak-tight fit therewith once attached with the attachment mechanism. In an example, the radially inward bias imposed on the collars are sufficient to enable entry into the mechanism by pushing action and retain and provide a leak-tight fit during use of the airway exchange bronchoscope construction. Also, the amount of radially inward bias imposed on the collars 120 and 122 is configured to permit removal of the bronchoscope and airway exchange catheter by pulling action relative to the attachment mechanism 110. Configured in this manner, the example attachment mechanism 110 facilitates easy and quick attachment/detachment between the airway exchange catheter and bronchoscope by respective push/pull action.

Airway-exchange procedures, in which one airway tube (e.g. endotracheal tube or tracheostomy tube) is exchanged for another, are a critical yet common element of Otolaryngology, Anesthesiology, and Critical Care. The morbidity and mortality associated with airway-exchange procedures has been well established. In the United States, the costs of complications from airway tube exchanges, such as accidental or unplanned extubations, have been estimated to be as high as $5 billion per year. During high-risk exchanges, airway-exchange catheters are often used to avoid loss of the airway. However, they are placed and exchanged blindly, and this has been shown to cause several complications including esophageal intubation, tension pneumothorax, gastric perforation, and airway trauma. Poor estimation of catheter depth is a known reason for this morbidity. Moreover, during an airway exchange, surgeons must switch between instruments, increasing procedural steps, time, and risk. There is also currently no way to visualize the distal end of an endotracheal tube or trach tube during an exchange while maintaining an airway exchange catheter in the airway.

A feature of airway exchange bronchoscope constructions as disclosed herein is that they have been specially designed and engineered to address the above-noted problems in high-risk airway-exchange procedures. A feature of airway exchange bronchoscope constructions as disclosed herein is that it facilitates placement of airway exchange catheters under visual guidance. Moreover, it increases efficiency by combining the air exchange catheter and bronchoscope into a single tool.

Airway Tube Exchange Procedure—In an example use application, an example bronchoscope construction as disclosed herein is passed through the preexisting airway tube and into the trachea to visualize the airway and carina. Next, the airway exchange catheter may be detached from the scope hand piece and remain in place in the airway tube and airway while the rest of the scope is withdrawn. The preexisting tube will be removed over the airway exchange catheter and the new tube will be introduced over the airway exchange catheter acting as a guidewire. Finally, the scope may be reinserted into the airway exchange catheter to visualize the carina and confirm correct placement in the airway. The entire device is then removed from the new airway tube, completing the airway exchange procedure.

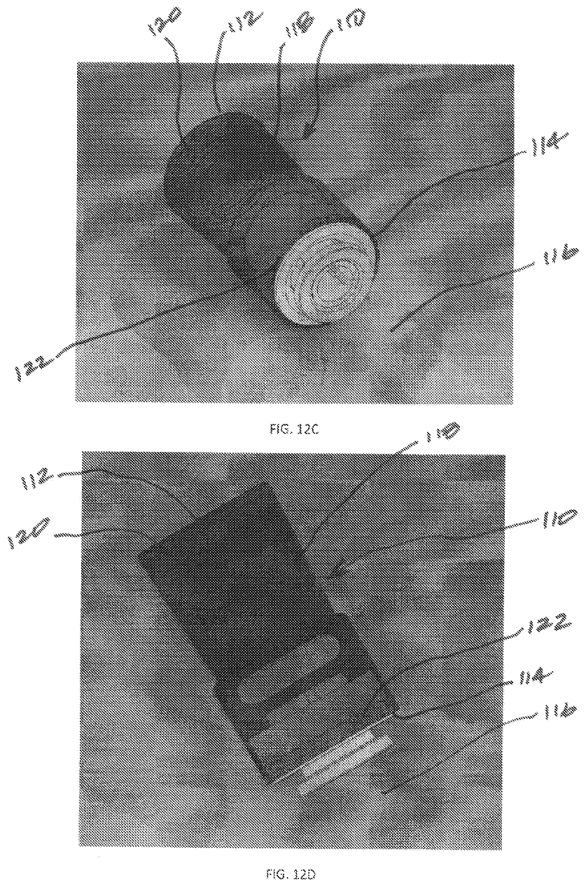

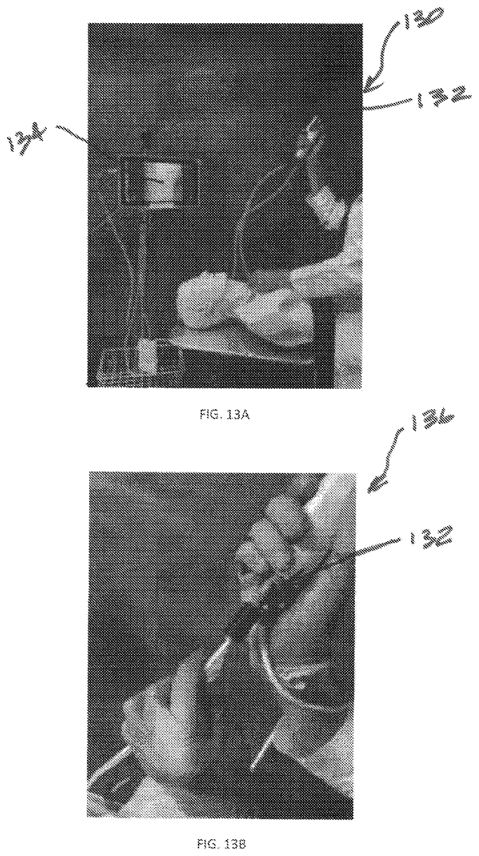

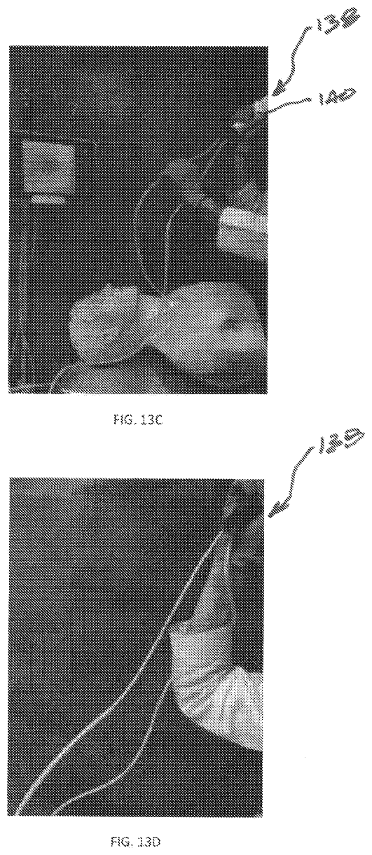

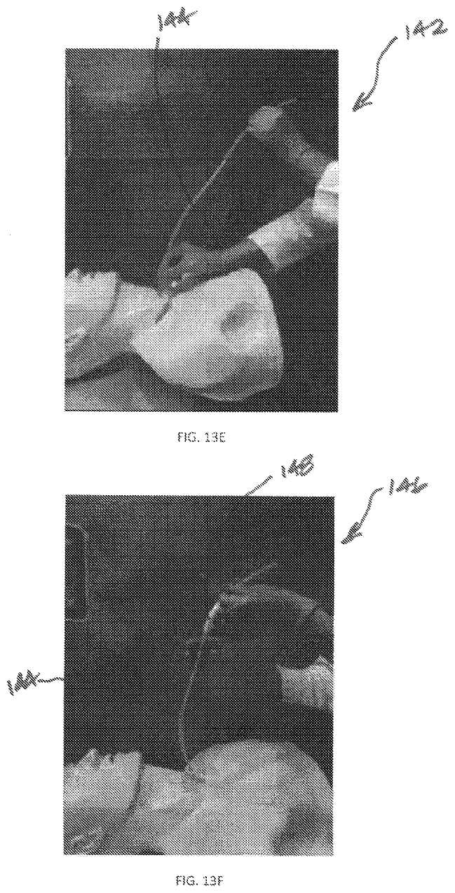

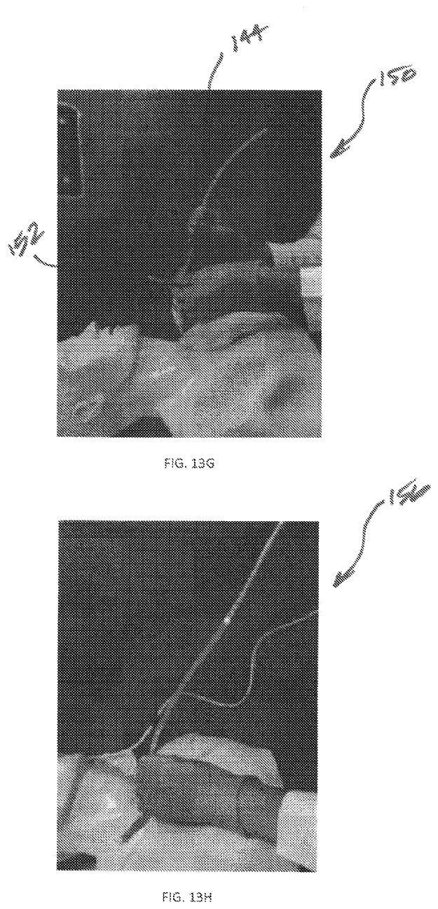

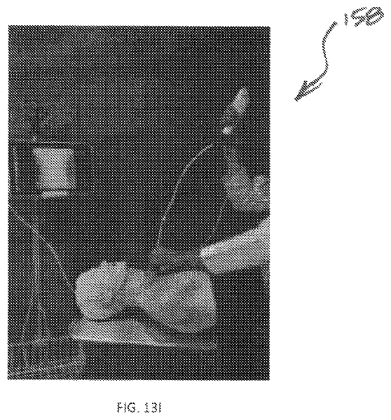

As briefly summarized above, a feature of bronchoscope constructions as disclosed herein is the ability to enable visual insertion and guidance of an airway exchange catheter into a patient's airway, e.g., during an airway tube exchange procedure. FIGS. 13A to 13I illustrate example steps showing use of an example bronchoscope construction as disclosed herein as used in an airway-exchange procedure. FIG. 13A illustrates the beginning 130 of an airway-exchange process or procedure, wherein the bronchoscope construction 132 as disclosed herein is introduced into an existing patient's airway tube to examine the tube's position, access the airway, and visualize the carina (using a display monitor 134 receiving a video image from the bronchoscope construction 132. FIG. 13B illustrates a stage 136 of the process where the airway exchange catheter of the bronchoscope construction is being placed at an appropriate depth in the patient under visualization (referring again to FIG. 13A). Once the desired placement position of the airway exchange catheter has been achieved, then the airway exchange catheter is removed from the bronchoscope construction, e.g., by use of the push-pull attachment mechanism (shown in FIG. 13B). FIGS. 13C and 13D illustrate a stage 138 of the process where the bronchoscope 140 is being decoupled and removed from the airway exchange catheter while the airway exchange catheter remains in the patient at the desired placement position as confirmed visually. FIG. 13E illustrates a stage 142 of the process where the airway-exchange catheter 144, placed under visual guidance, is now in proper position of exchange of airway tubes after the removal of the bronchoscope. FIG. 13F illustrate a stage 146 of the process where the old tracheostomy tube 148 is removed from the patient over the airway exchange catheter 144. FIG. 13G illustrates a stage 150 of the process where a new tracheostomy tube 152 is being introduced over the airway exchange catheter 144. FIG. 13H illustrates a stage 156 of the process where the bronchoscope is quickly and easily reintroduced into the airway exchange catheter to visually confirm successful placement in the airway. FIG. 13I illustrates a stage 158 where the airway exchange catheter is connected back with the bronchoscope via the attachment mechanism so that the carina can be visualized, confirming successful placement in the airway. In such example procedure, the bronchoscope construction may then be removed, ending the procedures. This is but one example procedure in which bronchoscope constructions as disclosed herein may be used to visually guide and verify desired placement of an airway in a patient, and it is to be understood that there may exist other procedures or instances where bronchoscope constructions as disclosed here may be used to provide visual guidance and confirmation of airway placement, and all such uses of bronchoscope constructions as disclosed herein is intended to be within the scope of this description.

A feature of airway exchange bronchoscope constructions as disclosed herein is they have been specifically configured to minimize steps and time associated with a procedure using both a bronchoscope and an airway exchange catheter. Another feature is that airway exchange bronchoscope constructions as disclosed herein enable insertion and placement of an airway exchange catheter under visual guidance.

Although example enclosure airway exchange bronchoscope constructions have been disclosed above and illustrated, those skilled in the art will readily appreciate that many modifications are possible in the example embodiments without materially departing from the intent and purpose of the example airway exchange bronchoscope constructions and their use as disclosed herein. Accordingly, all such modifications of airway exchange bronchoscope constructions and use of the same as disclosed herein are intended to be included within the scope of this disclosure as defined in the following claims.

What is claimed is:

1. An airway exchange bronchoscope construction comprising:
a bronchoscope comprising a body and a tube extending outwardly therefrom;
an airway exchange catheter comprising a tube that extends concentrically over the bronchoscope tube; and
an attachment mechanism that is configured to attach and detach the airway exchange catheter to the bronchoscope.

2. The airway exchange bronchoscope construction of claim 1, wherein the attachment mechanism comprises a rotatable element.

3. The airway exchange bronchoscope construction of claim 2, wherein the attachment mechanism is engage a feature of at least one of the bronchoscope and the airway exchange catheter.

4. The airway exchange bronchoscope construction of claim 1, wherein the attachment mechanism comprises one or more magnetic elements configured to promote attachment between the airway exchange catheter and the bronchoscope by magnetic force.

5. The airway exchange bronchoscope construction of claim 1, comprising a first magnetic element attached with the bronchoscope and a second magnetic element attached adjacent an end of the airway exchange catheter.

6. The airway exchange bronchoscope construction of claim 1, wherein the airway exchange catheter tube is configured to extend along a substantial entirety of the bronchoscope tube when the airway exchange catheter is attached with the bronchoscope.

7. The airway exchange bronchoscope construction of claim 1, wherein the attachment mechanism is configured to provide connection of the airway exchange catheter and the bronchoscope by ends of one or both of the airway exchange catheter and the bronchoscope being pushed into the attachment mechanism.

8. The airway exchange bronchoscope construction of claim 7, wherein the attachment mechanism is configured to provide detachment of the airway exchange catheter and the bronchoscope by ends of one or both of the airway exchange catheter and the bronchoscope being pulled out of the attachment mechanism.

9. A system for enabling visually guided placement of an airway exchange catheter into a patient comprising:
a bronchoscope comprising a body and a tube extending outwardly therefrom, the bronchoscope comprising a camera at one end, wherein the bronchoscope is configured to provide a video signal from the camera for visual monitoring;
an airway exchange catheter comprising a tube, wherein the airway exchange catheter tube is configured to accommodate placement of the bronchoscope tube therein; and
an attachment mechanism configured to facilitate removable connection of the bronchoscope with the airway exchange catheter when the bronchoscope tube is disposed inside of the airway exchange catheter tube.

10. The system of claim 9, wherein at least one of the bronchoscope or the airway exchange catheter tube is configured comprising a feature that registers with the attachment member.

11. The system of claim 9, wherein the attachment mechanism is configured to attach with and detach from the airway exchange catheter by respective push in and pull out action relative to the attachment mechanism.

12. The system of claim 9, wherein the attachment mechanism is configured to attach with and detach from the bronchoscope by respective push in and pull out action relative to the attachment mechanism.

13. The system of claim 9, wherein the bronchoscope tube is disposed inside of the airway exchange catheter.

14. The system of claim 13, wherein the attachment mechanism is interposed between the bronchoscope and the airway exchange catheter.

15. The system of claim 9, wherein the attachment mechanism is configured to attach with and detach from the airway exchange catheter by respective twisting action of one of the airway exchange catheter or attachment mechanism relative to the other of the relative to the other of the airway exchange catheter or attachment mechanism attachment mechanism.

16. A method for visually guiding placement of an airway inside of a patient comprising the steps of:
inserting an airway exchange catheter into an airway of a patient, wherein before or after the step of inserting, installing a tube of a bronchoscope inside a tube of the airway exchange catheter, wherein the bronchoscope comprises a camera at an end that has a field of vision directed out of an end of the airway exchange catheter tube that is disposed in the airway; and
viewing an image provided by the camera to visually confirm a desired placement position of the airway exchange catheter in the airway.

17. The method of claim 16, wherein the tube of the bronchoscope is installed inside the tube of the airway exchange catheter before the airway exchange catheter is inserted into the airway of the patient.

18. The method of claim 16, wherein during the step of installing, using an attachment mechanism to releasably connect the bronchoscope to the airway exchange catheter.

19. The method of claim 18, wherein at least one of the bronchoscope or the airway exchange catheter has a surface configured to register with the attachment mechanism.

20. The method of claim 16, wherein the step of viewing comprises using a video display that is operatively connected with the bronchoscope camera to receive a video signal.
